(19) United States
(12) Patent Application Publication
(10) Pub. No.: US 2004/0014694 A1
(43) Pub. Date: Jan. 22, 2004

(54) Use of docetaxel/doxorubicin/cyclophosphamide in adjuvant therapy

(76) Inventor: Hichem Chakroun, Courbevoie (FR)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/439,827

(22) Filed: May 16, 2003

Related U.S. Application Data
(60) Provisional application No. 60/380,850, filed on May 17, 2002.

(51) Int. Cl. A61K 31/704; A61K 31/66; A61K 31/337

(52) U.S. Cl. 514/34; 514/109; 514/449

(57) ABSTRACT

The present invention relates to a new method of adjuvant therapy in the treatment of early breast cancer, comprising administering six cycles of docetaxel, doxorubicin and cyclophosphamide to a patient in need thereof, wherein said dosages have a marked therapeutic effect when compared to other adjuvant therapies.

This application claims the benefit of U.S. Provisional Application No. 60/380,850, filed May 17, 2002.

### BACKGROUND OF THE INVENTION

1. Field of the Invention

This invention relates to a novel therapeutic use of a combination of taxotere with other antineoplastic agents in the adjuvant therapy of breast cancer.

2. Description of the State of the Art

The present invention relates more specifically to the use of docetaxel in combination with doxorubicin and cyclophosphamide as adjuvant therapy in the treatment of breast cancer, i.e., early stages of breast cancer immediately after diagnoses by routine screening, such as mammography or other commonly used methods.

Previous researchers have noted that docetaxel (TAXOTERE®) and other taxanes (such as TAXOL®, paclitaxel) are useful in the treatment of malignant neoplasms, such as solid tumors and other malignancies. European Patent EP 0 253 738 and International Patent Application WO 92/09589 describe a method of preparation of docetaxel. Generally, the doses, which vary depending on the patient, comprise between 60 and 200 mg/m² of docetaxel. Commonly, docetaxel is administered via intravenous route at doses of 60 to 100 mg/m² over 1 hour every 3 weeks (Textbook of Medical Oncology, Franco Cavelli et al., Martin Dunitz Ltd., p. 4623 (1997)).

Many clinical studies have confirmed the efficacy of docetaxel in treating many types of cancer, particularly breast, non-small cell lung, and ovarian cancer. Docetaxel's effects are shown in both first and second line therapies. The main mechanism of docetaxel's action is believed to be via enhancement of microtubule assembly and inhibition of the depolymerization of tubulin at the cellular level.

However, all treatments based on taxoid derivatives, including docetaxel, can show serious and troubling toxicities, such as myelosuppression, neutropenia, hypersensitivity, peripheral neuropathy, and fluid retention, among others (Fumoleau et al., Bull. Cancer, 82(8): 629-636 (1995)). When such toxicities appear, dosages of the drugs must be limited with a resulting limitation on the efficacy of the treatment.

Consequently, there is an unmet need in the art for pharmaceutical preparations and methods of treating cancer which enhance the activity of docetaxel without increasing the amount of the dosages administered and without increasing adverse side effects.

There is also an unmet need in the art for treatment of cancer which is still at the early stages and has not spread too much beyond the initial tumor site. In early breast cancer patients especially, there is a need for effective post-surgery adjuvant therapy, which will result in disease free survival or at least, an extension of the duration of progression free survival.

Anthracycline-based regimens, using e.g. doxorubicin or epirubicin, are standard adjuvant therapy in node positive breast cancer patients (French Epirubicin Study Group, J. Clin. Oncol., Vol. 6, 679-688 (1988)). However, in recent studies, docetaxel containing regimens have shown superior activity over standard regimens in metastatic breast cancer (Nabholtz, et al., Expert Opin. Pharmacother., Vol. 1(2), 187-206 (2000)).

In a few of the recent studies, where anthracycline-based regimens had failed, docetaxel based regimens showed improved efficacies. For instance, a phase III study compared docetaxel with mitomycin plus vinblastine (MV) in patients with metastatic breast cancer (MBC) progressing despite previous anthracycline containing chemotherapy. Docetaxel was found to be significantly superior to MV in terms of response, median time to progression (TTP), and survival (Nabholtz et al., J. Clin. Oncol., Vol. 17(5), 1413-1424 (1999)). A similar comparative study comparing the efficacy and tolerability of docetaxel to methotrexate and 5-fluorouracil (MF) in advanced breast cancer after anthracycline failure has been reported. Based on the response rate and the primary end point of TTP, docetaxel was found to be superior to sequential methotrexate and 5-fluoruracil in advanced breast cancer after anthracycline failure (Sjostrom et al., Euro. J. Cancer, Vol. 35 (8), 1194-1201 (1999)).

Similarly, there have been a few clinical studies which have shown the superior efficacy of docetaxel where the patients were treated with alkylating agent containing chemotherapy (e.g., cyclophosphamide, methotrexate, and 5-flurouracil (CMF) or its variants). For instance, a phase III clinical study compared docetaxel and doxorubicin in patients with metastatic breast cancer who had received previous alkylating agent containing chemotherapy (Chan et al., J. Clin. Oncol. Vol. 17 (8), 2341-2354 (1999)).

It has also been reported that various combination of drugs are useful in first line chemotherapy in treating advanced breast cancer. For example, in a phase III clinical study, a combination of doxorubicin and docetaxel (AT) has been compared with doxorubicin and cyclophosphamide (AC) as first line chemotherapy for metastatic breast cancer. This study showed that AT significantly improved TTP and overall response rate (ORR) compared with AC in patients with metastatic breast cancer (Nabholtz et al., J. Clin. Oncol., Vol. 21 (6), 968-975 (2003)).

Therefore, considering the efficacy of both docetaxel and doxorubicin in treating advanced breast cancer and their potential noncross-resistance, they have been combined with cyclophosphamide as a possible design for a more effective first line chemotherapy for metastatic breast cancer. The combination of the docetaxel, doxorubicin, and cyclophosphamide (TAC) was tested in a phase II trial in 54 patients. The results obtained therein indicated that TAC is active in first-line treatment of patients with breast cancer with manageable neutropenia side-effects and acceptable toxicity profile. Most importantly, this study showed no evidence of added cardiac toxicity for TAC compared with classical anthracycline containing combinations (Nabholtz et al., J. Clin Oncol., Vol. 19 (2), 314-321 (2001)).

However, there is still a need for developing combination chemotherapeutic drug regimens for adjuvant therapy in order to treat early stage breast cancer whereby not only the relapse of cancer is controlled but also increase overall survival (OS) of patients suffering from this debilitating disease. Thus it is an object of this invention to provide a combination regimen that satisfies this unmet need.

### SUMMARY OF THE INVENTION

Accordingly, the present invention embodies methods for treating breast cancer more preferably early breast cancer in an adjuvant setting comprising administering docetaxel, doxorubicin and cyclophosphamide (TAC) in amounts effective to reduce or eliminate the presence of cancer. The efficacy of this combination has been demonstrated over a period of thirty-three months in more than seven hundred human patients who demonstrated positive nodal involvement and were treated post-surgery with TAC.

Another aspect of the invention comprises new pharmaceutical kits and medicaments comprising docetaxel in combination with doxorubicin and cyclophosphamide for treating cancers.

Yet another aspect of the invention is concerned with schedules of administration of TAC for the adjuvant treatment of cancer wherein the individual drugs in the TAC combination are infused separately on the same day, once every three weeks. This cycle is repeated six times.

### BRIEF DESCRIPTION OF THE DRAWINGS

FIG. 1 illustrates disease free survival in each treatment group (intention-to-treat (ITT) population).

FIG. 2 illustrates overall survival in each treatment group (intention-to-treat (ITT) population).

FIG. 3 illustrates disease free survival by nodal status, 1-3 nodes vs. 4+ nodes.

FIG. 4 illustrates overall survival by nodal status, 1-3 nodes vs. 4+ nodes.

FIGS. 5A and 5B illustrate disease free survival by hormonal status.

FIGS. 6A and 6B illustrate overall survival by hormonal status.

FIGS. 7A and 7B illustrate disease free survival by HER2 status.

### DESCRIPTION OF THE PREFERRED EMBODIMENTS

Selected term definitions used herein and in accompanying drawings are as follows:

“Adjuvant therapy” as stated herein shall have the generally accepted meaning. It shall also refer to chemotherapy started within but no greater than 60 days from surgery. It shall also mean that treating asymptomatic patients, i.e., healthy patients having early stages of breast cancer who are diagnosed by routine physical screenings, such as mammograms, breast examinations, etc.

“AT” refers to Adriamycin/Taxotere;

“docetaxel” refers to the active ingredient of TAXOTERE® or TAXOTERE® itself;

“doxorubicin” refers to the active ingredient of ADRIAMYCIN® or ADRIAMYCIN® itself.

“ER” refers to estrogen receptor;

“FAC” refers to the combination of 5-fluorouracil, doxorubicin and cyclophosphamide;

“HER2” refers to a transmembrane receptor tyrosine kinase with partial homology with the epidermal growth factor 2 receptor, both of which receptors belong to the type 1 tyrosine kinase receptor superfamily;

“KPS” refers to Karnovsky Performance Status which is an index of a patient's physical condition;

“MF” refers to Methotrexate/5-Fluorouracil;

“MV” refers to Mitomycin/Vinblastin combination;

“PR” refers to progesterone receptor;

“TAC” refers to the combination of TAXOTERE© (docetaxel), ADRIAMYCIN (doxorubicin) and cyclophosphamide;

and

“drug” or “drugs” refers to the above-mentioned active ingredients or medicaments or pharmaceutical preparations containing them.

As discussed above, considering the efficacy of both docetaxel and doxorubicin in treating advanced breast cancer and their potential noncross-resistance, it was decided to combine them with cyclophosphamide as a possible design for a more effective adjuvant chemotherapy for early breast cancer. The combination of the docetaxel, doxorubicin, and cyclophosphamide (TAC) was tested in a phase III trial in 20 countries by more than 112 investigators. Surprisingly, the results which are elaborated below show that the combination used as adjuvant therapy enhances the effect of docetaxel without increasing its dosage and results in increased survival for early breast cancer patients.

The inventor of the present invention has demonstrated via clinical trials, that TAC dosages in particular manifest an unexpected and strong therapeutic effect on the treatment of neoplastic diseases, particularly breast cancers, and more particularly, in early breast cancers in which ER/PR and HER2 are overexpressed. Generally, according to the invention, docetaxel is administered in a dosage of 75 mg/m², doxorubicin in a dosage of 50 mg/m² and cyclophosphamide in a dosage of 500 mg/m², once every three weeks. This cycle is repeated six times. This regimen is further compared with a FAC dosage comprising 5-flurouracil administered in a dosage of 500 mg/m², doxorubicin in a dosage of 50 mg/m² and cyclophosphamide in a dosage of 500 mg/m², once every three weeks. This cycle is again repeated six times.

Docetaxel alone, in several in-house proprietary studies, gave overall response rates of 40 to 43% (in second line therapy at a dose of 100 mg/m²), 48% (in first line therapy at a dose of 75 mg/m²) and 61% (in first line therapy at a dose of 100 mg/m²).

In comparison, in the example below, 75 mg/m² of docetaxel administered in combination with 50 mg/m² of doxorubicin and 500 mg/m² of cyclophosphamide resulted in an 82% response rate.

According to the invention, the new use of docetaxel as a component of TAC is very advantageous for treating cancers of the breast, ovary, and lung; still more preferably, the new use of docetaxel is particularly suitable for treating early breast cancer.

The safety and the efficacy of the combination of docetaxel, doxorubicin and cyclophosphamide was tested in patients according to the following protocol:

Patients were eligible for the study if they had histologically proven breast cancer, definitive surgery with axillary lymph node dissection (greater than or equal to 6 lymph nodes), a period of 60 days or less between surgery and randomization, stage 1 to 3 cancer, at least one node that was positive for cancer, were 70 years old or less, had a KPS index greater than or equal to 80% and had normal bone marrow, liver, renal and cardiac function.

One thousand, four hundred and ninety-one patients were accepted into the study. Seven hundred and forty-five received TAC as adjuvant therapy and seven hundred and forty-six received FAC. The TAC patients had a median age of 49 years, 51% were premenopausal, and 60% had had a mastectomy. Sixty-eight % had had radiotherapy and 68% had taken tamoxifen. The patient characteristics for the FAC group were similar (See Table 1).

TABLE 1
Patient Characteristics

| | TAC | FAC |
|---|---|---|
| Randomized (n = 1,491) | n = 745 | n = 746 |
| Median Age | 49 | 49 |
| Median KPS | 100% | 100% |
| Premenopausal | 51% | 50% |
| Mastectomy | 60% | 59% |
| Radiotherapy | 68% | 71% |
| Tamoxifen | 68% | 69% |

Of the seven hundred and forty-five TAC patients, 62% had 1-3 cancer-positive nodes, 30% had 4 to 10 positive nodes and 8% had more than 10 positive nodes. In 40% of the patients, the size of the tumor was 2 cm or less, in 53%, the size of the tumor was more than 2 cm but equal to or less than 5 cm., and in 7% of the patients, the tumor was larger than 5 cm. Sixty-nine per cent of the patients had overexpressing ER or PR tumors and 19% had overexpressing HER2+(FISH) tumors. Again, the tumor characteristics of the FAC group were comparable. See Table 2.

The primary endpoint of this Phase III study was to facilitate disease free survival while secondary endpoints were overall survival, toxicity, quality of life and monitoring pathological and molecular markers.

TABLE 2
Tumor Characteristics

| | TAC | FAC |
|---|---|---|
| | n = 745 | n = 746 |
| Nodal Status | & | % |
| 1-3 | 62 | 62 |
| 4-10 | 30 | 31 |
| >10 | 8 | 7 |
| Tumor Size (cm) | | |
| ≦2 | 40 | 43 |
| >2 and ≦5 | 53 | 51 |
| >5 | 7 | 6 |
| ER and/or PR + | 69 | 69 |
| HER2+ (FISH) | 19 | 20 |

Post-TAC and post-FAC treatment included 1) radiation therapy for all patients with breast conserving surgery in accordance with each study center's guidelines after mastectomy; and 2) tamoxifen (20 mg/day for 5 years) for those patients with ER or PR positive tumors.

The present invention also relates, therefore, to preparation of pharmaceutical kits containing the combinations according to the invention.

The constituents of which the combination are composed may be administered separately, or spaced out over a period of time so as to obtain the maximum efficacy of the combination; it being possible for each administration to vary in its duration from a rapid administration to a continuous perfusion.

The compositions in the kit according to the invention are preferably compositions which can be administered parentally. The compositions for parental administration are generally pharmaceutically acceptable, sterile solutions or suspensions which may optionally be prepared as required at the time of use. For the preparation of non-aqueous solutions or suspensions, natural vegetable oils such as olive oil, sesame oil or liquid petroleum or injectable organic esters such as ethyl oleate may be used. The sterile aqueous solutions can consist of a solution of the product in water. The aqueous solutions are suitable for intravenous administration provided the pH is appropriately adjusted and the solution is made isotonic, for example with a sufficient amount of sodium chloride or glucose. The sterilization may be carried out by heating or by any other means which does not adversely affect the composition. The combinations may also take the form of liposomes or the form of an association with carriers as cyclodextrins or polyethylene glycols.

The compositions for oral or intraperitoneal administration are preferably aqueous suspensions or solutions.

The Example below illustrates the new use of docetaxel according to the invention without limiting it.

### EXAMPLE

Dexamethasone, 8 mg was given twice daily as a premedication for 3 days. The combination adjuvant therapy was then administered on Day 4. One group of patients received docetaxel, doxorubicin and cyclophosphamide (TAC) administered intravenously in that order. Another group of patients received 5-FU, doxorubicin, and cyclophosphamide (FAC) administered intravenously in that order. Prophylactic Cipro was then given to both groups on days 5-14 in a dose of 500 mg twice daily. This course of drugs was repeated every three weeks for six cycles.

Six hundred and seventy-nine patients (91%) completed six cycles of TAC adjuvant therapy followed by the postchemical therapy regimens described above. The median total dose per patient over the six cycles was 446 mg/m² of docetaxel, 297 mg/m² of doxorubicin, and 2978 mg/m² of cyclophosphamide. See Table 3.

TABLE 3
Exposure to Treatment

| | TAC | FAC |
|---|---|---|
| | N = 745 | n = 746 |
| Completed 6 cycles | 679 | 711 |
| | (91%) | (96%) |
| Relative dose Intensity | | |
| Median | 0.98 | 0.97 |
| >0.90 | 89% | 84% |
| Median total dose mg/m² | | |
| Docetaxel | 446 | — |
| Doxorubicin | 297 | 298 |
| Cyclophosphamide | 2978 | 2985 |
| 5FU | — | 2985 |

Seven hundred and eleven patients (96%) completed six cycles of FAC adjuvant therapy followed by the postchemical therapy regimens described above. The median total dose per patient over the six cycles was 2985 mg/m² of 5-FU, 298 mg/m² of doxorubicin, and 2985 mg/m² of cyclophosphamide. Id.

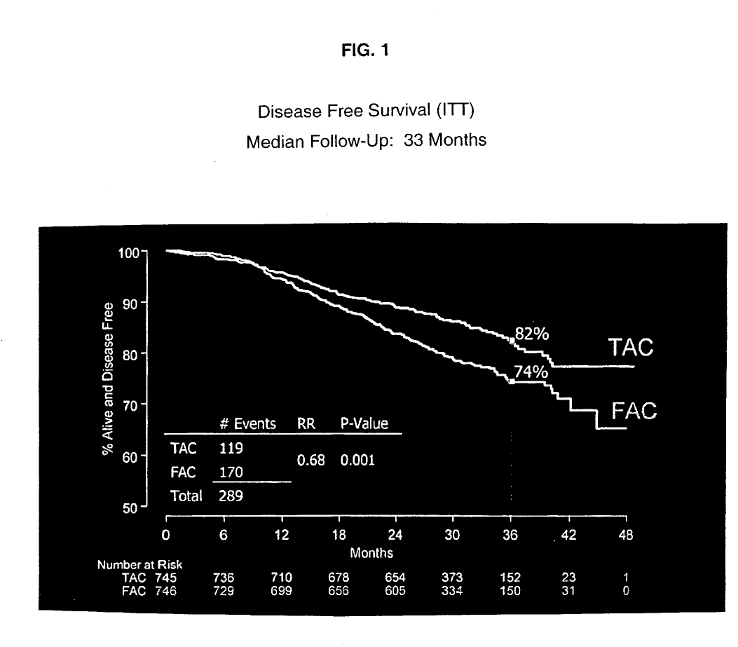

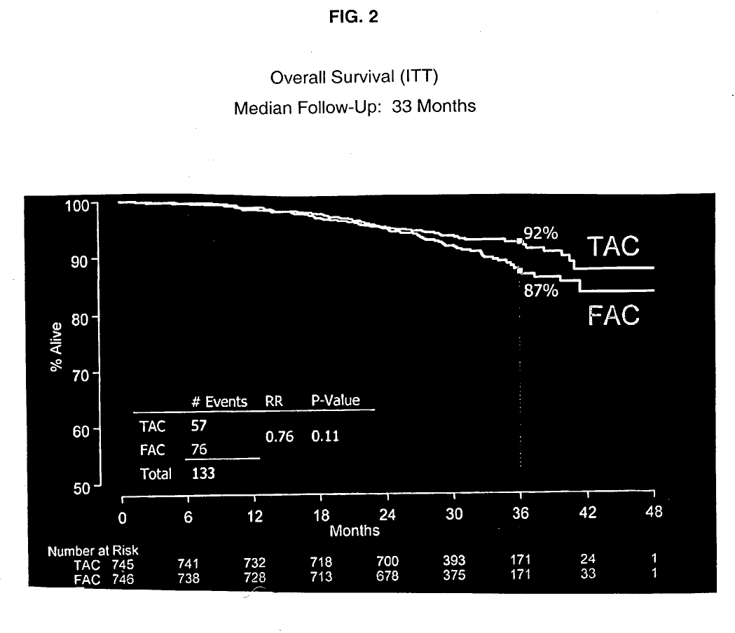

For purposes of statistical analyses, the protocols for assessing both the disease free survival (DFS) and the overall survival (OS) are defined as follows. The first planned analysis was for three years. The cohort used was intent to treat (ITT). The main analysis involved log ranking test stratified by nodal status and the p-values were not adjusted for interim analysis. The confirmatory analyses are unadjusted and use a multivariate Cox model. Thirty-three months after adjuvant therapy, 82% of the TAC group vs. 74% of the FAC group were alive and disease free (FIG. 1). At the same time, the overall survival of the TAC group was 92% vs. 87% for the FAC group (FIG. 2).

Table 4 summarizes the confirmatory analyses used in arriving the DFS and OS results respectively. Table 5 summarizes sites of first events.

TABLE 4
Comfirmatory Analyses

| | | Risk Ratio (RR) | | p | |
|---|---|---|---|---|---|
| Analysis | Cohort | DFS | OS | DFS | OS |
| Stratify on Nodes | ITT | 0.68 | 0.76 | 0.001 | 0.11 |
| | | (0.54-0.86) | (0.54-1.07) | | |
| Unadjusted | ITT | 0.67 | 0.75 | 0.0008 | 0.10 |
| | | (0.53-0.85) | (0.53-1.06) | | |
| Cox Model* | ITT | 0.64 | 0.71 | 0.0002 | 0.049 |
| | | (0.50-0.81) | (0.50-1.00) | | |

*Controls for nodes, age, tumor size, histology, ER/PR, HER2

TABLE 5
Sites of First Events

| | TAC (N) | FAC (N) |
|---|---|---|
| Distant | 80 | 119 |
| Local/Regional | 23 | 31 |
| Contralateral | 3 | 6 |
| Other 2^{nd} Primary | 6 | 10 |
| Death NED | 7 | 4 |
| Total | 119 | 170 |

Results by Nodal Status

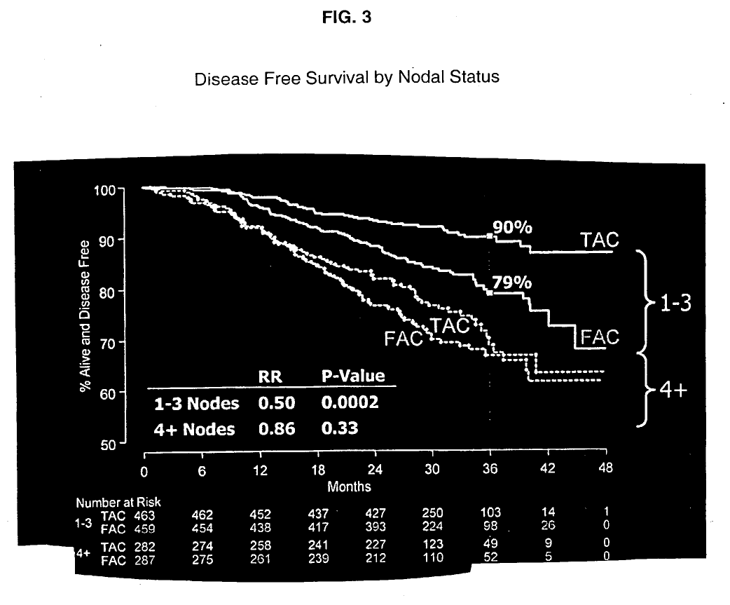

If disease free survival of the TAC and FAC groups is compared by nodal status, 90% of patients with 1-3 positive nodes who received TAC were alive and disease free at 33 months after therapy vs. 79% of the FAC group. There was no statistical difference between the two adjuvant therapies in patients with 4 nodes, although 69% of patents receiving TAC therapy were alive and disease free at 36 months compared to 67% who received FAC. See FIG. 3.

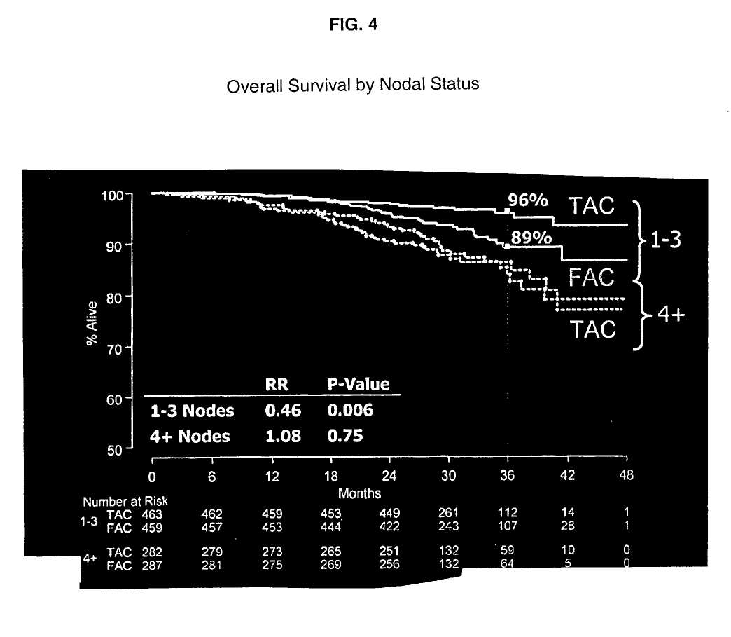

The overall survival rate for patients with 1-3 positive nodes was 96% for TAC and 89% for FAC. Again, there was no statistical difference between the two therapies in patients with 4 or more positive nodes, although again more TAC patients (86%) survived than FAC patients (84%). See FIG. 4.

Results by Hormonal Status

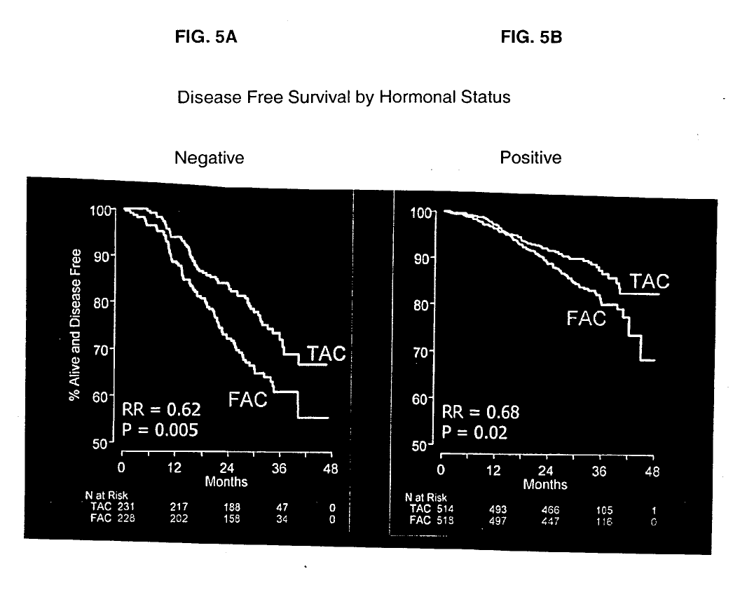

In patients with negative ER/PR tumors, the disease free survival rate was about 70% in those who had received TAC adjuvant therapy and about 62% in those receiving FAC. In patients with positive ER/PR nodes, the disease free survival rate among those who received TAC was about 88% vs. 82% in those who received FAC. See FIGS. 5A and 5B.

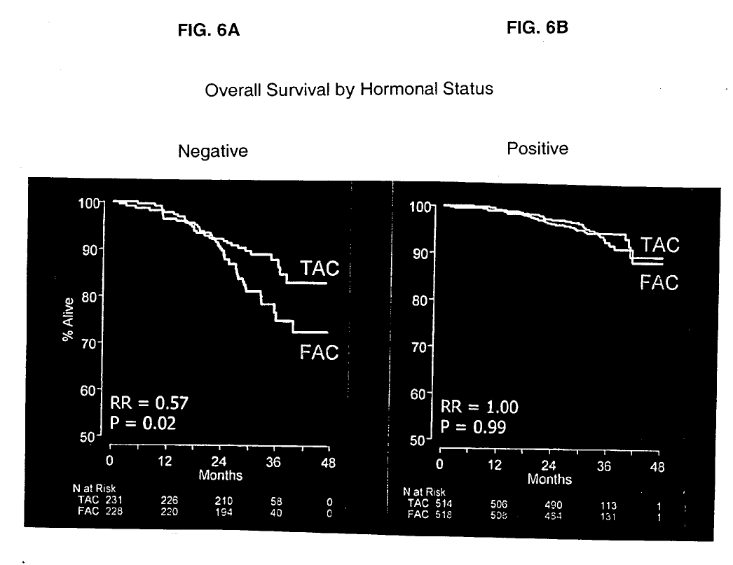

If one calculates overall survival by hormonal status, about 83% of TAC recipients with negative ER/PR tumors survived vs. about 72% of FAC recipients. Among patients with positive tumors, about 90% of TAC recipients survived vs. about 88% of FAC recipients. See FIGS. 6A and 6B.

Results by HER2 Status

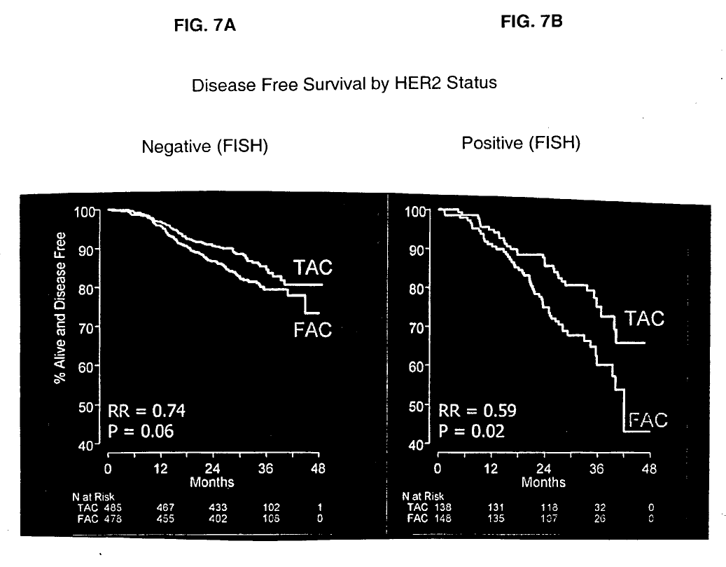

In patients with negative HER2 tumors, the disease free survival rate at 33 months was about 86% in those who had received TAC adjuvant therapy and about 80% in those receiving FAC. In patients with positive HER2 tumors, the disease free survival rate among those who received TAC was about 75% vs. 60% in those who received FAC. See FIGS. 7A and 7B.

Safety

Hematologic toxic events are summarized in Table 6. The incidence of ANC (<1000) was higher with TAC than with FAC (65.1% v 49%). Febrile neutropenia occurred more frequently with TAC than with FAC, without increased incidence of infection and no septic deaths. Other toxicities were acceptable and manageable in both arms as summarized in Tables 6 and 7. Also summarized in Table 6 are the data obtained for anemia (grade ¾) and thrombocytopenia (grade ¾) both of which showed higher occurrences for TAC than for FAC.

TABLE 6
Hematological Toxicity

| | TAC | FAC |
|---|---|---|
| | n = 744 | n = 736 |
| Treated (n = 1480) | % | % |
| ANC < 1000^{a} | 65.1 | 49.0 |
| Febrile Neutropenia^{b} | 23.9 | 2.4 |
| Infection (Grade 3/4) | 3.1 | 1.5 |
| Septic Death | 0 | 0 |
| Anemia (Grade 3/4) | 4.8^{c} | 2.2 |
| Thrombocytopenia (Grade 3/4) | 2.4 | 1.8 |

^{a}Protocol required blood counts every 3 weeks
^{b}Grade 4 neutropenia at time of grade ≧ 2 fever and i.v. antibiotics
^{c}p ≦ 0.05

Nonhematological adverse events are listed in Table 7. The occurrences of nausea and vomiting were higher with FAC than with TAC. Whereas the occurrences of other gastrointestinal toxicities such as diarrhea and stomatitis were somewhat higher with TAC than with FAC. Asthenia was also more frequent with TAC (11.2 v 5.3). CHF occurred in 1.6% of patients receiving TAC and 0.7% receiving FAC.

Based on this data, the combination of docetaxel, doxorubicin and cyclophosphamide as adjuvant therapy is well-tolerated and results in a significant advantage over 5FU, doxorubicin and cyclophosphamide as adjuvant therapy. If one measures disease free survival, at 33 months, TAC provided over FAC a 32% reduction in deaths overall, a 50% reduction in deaths where the patients had 1-3 positive nodes, a 38% reduction in deaths where the hormonal status of the tumor was negative and a 32% reduction where the hormonal status was positive. See Table 8.

TABLE 7
Non-Hematological Toxicity
Grade 3/4—Incidence >1%

| | TAC | FAC |
|---|---|---|
| | n = 744 | n = 736 |
| | % | % |
| Nausea | 5.1 | 9.5^{a} |
| Vomiting | 4.3 | 7.3^{a} |
| Diarrhea | 3.4^{a} | 1.0 |
| Stomatitis | 7.1^{a} | 2.0 |
| Asthenia | 11.2^{a} | 5.3 |
| CHF | 1.6 | 0.7 |
| Premenopausal patients | n = 383 | n = 375 |
| Amenorrhea | 51.4^{a} | 32.8 |

^{a}p ≦ 0.05

TABLE 8
Conclusions
At 33 months median follow-up, TAC provides over FAC:
Primary Endpoint: Disease-Free Survival

| | | |
|---|---|---|
| Overall: | | 32% reduction (p = 0.001) |
| By nodal status | 1-3: | 50% reduction (p = 0.0002) |
| | 4+: | No difference |
| By hormonal status | HR−: | 38% reduction (p = 0.005) |
| | HR+: | 32% reduction (p = 0.02) |

Secondary Endpoint: Overall Survival

| | | |
|---|---|---|
| Overall: | | 24% reduction (p = 0.11) |
| By nodal status | 1-3: | 54% reduction (p = 0.006) |
| | 4+: | No difference |

If one measures overall survival, there was a 24% reduction in deaths of those receiving TAC adjuvant therapy and a 54% reduction in those patients with 1 to 3 positive nodes. Id.

The difference between TAC and FAC is the presence of docetaxel rather than 5-FU. These statistics prove conclusively that the observed benefit of docetaxel in combination with doxorubicin and cyclophophamide is large enough to be of clinical value in the adjuvant treatment of node positive breast cancer patients.

The described embodiments are to be considered in all respects as illustrative only and not restrictive. The scope of the invention is, therefore, indicated by the appended claims rather than by the foregoing description. All changes which come within the meaning and range of equivalency of the claims are to be embraced within their scope.

What is claimed is:

1. A method of treating early breast cancer comprising administering docetaxel, doxorubicin and cyclophosphamide as adjuvant therapy to a patient in need thereof.

2. The method according to claim 1, wherein said docetaxel, doxorubicin and cyclophosphamide are administered separately.

3. The method according to claim 1, wherein said patient is having at least one cancer positive node.

4. The method according to claim 1, wherein said patient is having less than about 3 cancer positive nodes.

5. The method according to claim 1, wherein said patient is having from about 4 to about 10 nodes.

6. The method according to claim 1, wherein in said breast cancer the tumor size is about 2 cm.

7. The method according to claim 1, wherein in said breast cancer the tumor size is from about 2 cm to about 5 cm.

8. The method according to claim 1, wherein in said breast cancer, ER, PR and/or HER2 are overexpressed.

9. The method according to claim 2, wherein said administration of docetaxel, doxorubicin and cyclophosphamide is via an intravenous route.

10. The method according to claim 2, wherein docetaxel, doxorubicin and cyclophosphamide are administered once every 3 weeks.

11. The method according to claim 10, wherein docetaxel is administered at a dose of approximately 75 mg/m² per cycle.

12. The method according to claim 10, wherein doxorubicin is administered at a dose of approximately 50 mg/m² per cycle.

13. The method according to claim 10, wherein cyclophosphamide is administered at a dose of approximately 500 mg/m² per cycle.

14. A process for producing a pharmaceutical kit comprising dispensing in separate containers therapeutically effective amounts of docetaxel, doxorubicin and cyclophosphamide.

15. The process according to claim 14, wherein said kit is comprising sufficient quantities of docetaxel to administer to a patient at a dose of approximately 75 mg/m², sufficient quantities of doxorubicin to administer to a patient at a dose of approximately 50 mg/m², and sufficient quantities of cyclophosphamide to administer to a patient at a dose of approximately 500 mg/m².

16. The process according to claim 14, wherein said kit is suitable for the treatment of early breast cancer in a patient.

17. The process according to claim 16, wherein docetaxel, doxorubicin and cyclophosphamide are administered sequentially to a patient in need of said treatment.

18. A process for producing a pharmaceutical kit comprising dispensing in separate containers therapeutically effective amounts of docetaxel, doxorubicin and cyclophosphamide suitable for the treatment of early breast cancer in a patient.

19. The process according to claim 18, wherein said kit is comprising sufficient quantities of docetaxel to administer to a patient at a dose of approximately 75 mg/m², sufficient quantities of doxorubicin to administer to a patient at a dose of approximately 50 mg/m², and sufficient quantities of cyclophosphamide to administer to a patient at a dose of approximately 500 mg/m².

20. The process according to claim 18, wherein docetaxel, doxorubicin and cyclophosphamide are administered sequentially to a patient in need of said treatment.

21. The process according to claim 20, wherein said patient is having at least one cancer positive node.

22. The process according to claim 20, wherein said patient is having less than about 3 cancer positive nodes.

23. The process according to claim 20, wherein said patient is having from about 4 to about 10 cancer positive nodes.
